(19) United States
(12) Patent Application Publication
(10) Pub. No.: US 2024/0277742 A1
(43) Pub. Date: Aug. 22, 2024

(54) TRANSLATION INHIBITOR WITHOUT OCCUPYING RIBOSOME RESOURCES AS ANTI-TUMOR DRUG

(71) Applicant: CHI BIOTECH CO., LTD., Shenzhen (CN)

(72) Inventors: Gong ZHANG, Shenzhen (CN); Yang CHEN, Shenzhen (CN); Zhuo YU, Shenzhen (CN)

(21) Appl. No.: 18/442,146

(22) Filed: Feb. 15, 2024

Related U.S. Application Data
(63) Continuation of application No. PCT/CN2022/106988, filed on Jul. 21, 2022.

(30) Foreign Application Priority Data
Jan. 24, 2022 (CN) 202210082654.8

(51) Int. Cl. A61K 31/7034; A61K 31/235; A61K 31/58; A61P 35/00

(52) U.S. Cl. CPC A61K 31/7034; A61K 31/235; A61K 31/58; A61P 35/00

(57) ABSTRACT

Provided is a translation inhibitor without occupying ribosome resources. The translation inhibitor without occupying ribosome resources shows a good anti-tumor effect and is highly safe, and can be used in preparation of an anti-tumor drug.

### CROSS-REFERENCE TO RELATED APPLICATION

This application is a continuation of PCT application serial no. PCT/CN2022/106988, filed on Jul. 21, 2022, which claims the priority and benefit of Chinese patent application serial no. 202210082654.8, filed on Jan. 24, 2022. The entireties of PCT application serial no. PCT/CN2022/106988 and Chinese patent application serial no. 202210082654.8 are hereby incorporated by reference herein and made a part of this specification.

### TECHNICAL FIELD

The present invention belongs to the field of medicine and relates to an anti-tumor drug, in particular to a translation inhibitory molecule without occupying ribosome resources for use in anti-tumor drugs.

### BACKGROUND

The unlimited proliferation, migration and invasion of tumors are inseparable from vigorous protein synthesis (i.e., translation process). There are many classic cancer-promoting pathways in tumor cells, such as MAPK pathway, mTOR pathway, all of which enhance translation downstream. By up-regulating the translation efficiency of p90RSK in cancer cells, the translation initiation pathway also can be self-activated through positive feedback, thereby maintaining the high activity of their own translation system (Wang et al., Translating mRNAs strongly correlate to proteins in a multivariate manner and their translation ratios are phenotype specific, Nucleic Acids Research, 2013, 41(9): 4743-54). Therefore, inhibiting the translation system has long been considered as a possible solution to inhibit tumor cells. Currently, rapamycin, temsirolmus, everolimus and other drugs are used clinically as anti-cancer drugs. They all target mTOR and have certain curative effects. However, they are prone to failure for the reason as mentioned above that there are many pathways in cancer cells that can up-regulate the translation system. For example, p90RSK and p70S6K can bypass mTOR and can therefore be used by tumor cells to cause resistance to these drugs. So people hope to directly inhibit the translation system itself. Decades ago, the classic translation inhibitor cycloheximide was used to treat cancer, but it had severe side effects and was never used as a first-line drug. It is no longer used clinically. Harringtonine, also known as omacetaxine, has also been clinically tried to treat cancer in recent years, but it cannot be used in treatment of solid tumors because of its severe side effects.

Therefore, it is desired to provide a new translation inhibitor to solve the above technical problems.

### SUMMARY

The present invention provides a translation inhibitor, in particular to a translation inhibitor without occupying ribosome resources. The translation inhibitor only prevents the translation initiation process and does not jam the ribosomes on the mRNA, does not interfere with the translation elongation function of the assembled ribosomes and does not significantly promote their dissociation. Preferably, the translation inhibitor without occupying ribosome resources is selected from methyl aurintricarboxylate, Kasugamycin and Hippuristanol, or a prodrug of one of methyl aurintricarboxylate, Kasugamycin and Hippuristanol or one or more of pharmaceutically acceptable salts of one of methyl aurintricarboxylate, Kasugamycin and Hippuristanol. Preferably, the translation inhibitor without occupying ribosome resources is methyl aurintricarboxylate, Kasugamycin or Hippuristanol, or a prodrug of one of methyl aurintricarboxylate, Kasugamycin and Hippuristanol, or a pharmaceutically acceptable salt of one of methyl aurintricarboxylate, Kasugamycin and Hippuristanol. More preferably, the pharmaceutically acceptable salt is an ammonium salt, a sodium salt, a potassium salt or a hydrochloride salt.

The translation inhibitor without occupying ribosome resources according to the present invention can be used in preparation of an anti-tumor drug, preferably, in preparation of an anti-malignant tumor drug, especially against lung cancer, ovarian cancer, liver cancer or breast cancer.

In some cases, the translation inhibitor without occupying ribosome resources can also be used in combination with other known anti-tumor drugs.

The present invention further provides a pharmaceutical composition comprising a translation inhibitor without occupying ribosome resources and a pharmaceutically acceptable carrier. Preferably, in the pharmaceutical composition, the translation inhibitor without occupying ribosome resources is selected from methyl aurintricarboxylate, kasugamycin and Hippuristanol, or a prodrug of one of methyl aurintricarboxylate, Kasugamycin and Hippuristanol or one or more of pharmaceutically acceptable salts of one of methyl aurintricarboxylate, Kasugamycin and Hippuristanol.

In some cases, the pharmaceutically acceptable carrier includes, but is not limited to, a diluent, a filler, a binder, a wetting agent, a disintegrant, an absorption enhancer, a surfactant, an adsorbent carrier, and a lubricant. The pharmaceutical composition can be formulated into liquid preparation forms such as tablets, capsules, powders, granules, lozenges, suppositories, oral liquids or sterile parenteral suspensions, and injection forms such as large- or small-volume injections and freeze-dried powders.

The present invention further provides an anti-tumor drug kit, comprising the pharmaceutical composition described above and one or more other anti-tumor drugs.

The translation inhibitor without occupying ribosome resources according to the present invention can effectively inhibit the malignant phenotype of tumors, and at the same time has high safety. It can be used to prepare anti-tumor drugs and has broad application prospects in preventing or treating tumors.

### BRIEF DESCRIPTION OF DRAWINGS

FIG. 1 shows chemical structures of some translation inhibitors without occupying ribosome resources, A: Methyl aurintricarboxylate; B: Kasugamycin; C: Hippuristanol.

FIG. 2 shows experimental results of some active molecules without occupying ribosome resources.

FIG. 3 shows anti-tumor activity and safety test of methyl aurintricarboxylate on lung cancer cells, where A: cell migration; B: cell invasion; C: cell apoptosis; D: cell colony formation; and E: cytotoxicity assays.

FIGS. 4A-4I shows the effect of methyl aurintricarboxylate on apoptosis of ovarian cancer cells.

FIG. 5 shows the inhibitory effect of methyl aurintricarboxylate on proliferation of liver cancer cells.

FIG. 6 shows the inhibitory effect of Kasugamycin on proliferation of breast cancer cells.

FIG. 7 shows the anti-tumor activity of Kasugamycin in a nude-mouse lung cancer xenograft model.

FIG. 8 shows the inhibitory effect of Hippuristanol on proliferation of breast cancer cells, where Hipp refers to Hippuristanol.

FIG. 9 shows the anti-tumor activity of Hippuristanol in a nude-mouse lung cancer xenograft model.

### DESCRIPTION OF EMBODIMENTS

The methods and techniques of the present invention are generally performed according to conventional methods known in the art, unless otherwise stated. Nomenclature, and experimental methods and techniques related to biology, pharmacy, medicine, and chemistry described herein are known and commonly used in the art. Standard techniques are used for cell and tissue-related culture and testing methods, pharmaceutical preparation, formulation and delivery, and patient treatment.

Unless otherwise stated, scientific and technical terms used herein shall have the meaning commonly understood by those skilled in the art. However, the following terms are defined as follows:

“Translation”, as used herein, refers to eukaryotic translation, which is the biological process in which messenger RNA is translated into proteins in eukaryotes. It consists of four phases: initiation, extension, termination, and recycling. Translation initiation is the first and most complex step in the translation process. In eukaryotes, this process can be divided into three stages: First, a variety of translation initiation factors and related proteins bind to the small subunit of 40S ribosome, and then to the initiate tRNA methionine, thereby forming a 43S pre-initiation complex; then, the pre-initiation complex binds to the 5′ end of the activated mRNA and moves from the 5′ end to the 3′ end on the 5′ untranslated sequence until it finds the correct start codon (typically, the first AUG) and forms a 48S complex; then, the large subunit of the 60S ribosome binds to the 48S complex to finally form an 80S initiation complex, which is ready to initiate translation. Each step in the initiation process requires the participation of multiple eukaryotic initiation factors.

“Translation inhibitor” broadly refers to active substances that inhibit or hinder the translation process or translation factors. In the present application, it refers to active molecules that inhibit the initiation or elongation of translation.

“Ribosome” is an organelle in cells. It is formed by the combination of two subunits, one large and one small. Its main components are intertwined RNA (also known as “ribosomal RNA” or “rRNA”) and proteins (also known as “ribosomal protein” or “RP”). The ribosome is the site of protein synthesis in cells. It can read genetic information contained in the nucleotide sequence of messenger RNA and convert the genetic information into the sequence information of amino acids in proteins to synthesize proteins.

“Without occupying ribosome resources” means that during the process of inhibiting translation, the ribosome is not kept on the mRNA, the large and small subunits of the ribosome are still free and only the assembly of the initiation complex is affected. It does not affect ribosomes that have entered the elongation state of translation, does not prevent their elongation process, and does not actively dissociate intact ribosomes.

“Translation inhibitor without occupying ribosome resources” refers to an inhibitor that prevents the translation initiation process, does not keep ribosomes on mRNA, does not interfere with the translation elongation function of assembled ribosomes, and does not significantly promote their dissociation

“Tumor” is a new organism formed when a certain cell in a local tissue of the body loses normal control of its growth at the genetic level under the action of various carcinogenic factors, resulting in abnormal clonal proliferation. Tumors are generally divided into two categories: benign and malignant. Malignant tumors are often called cancer. Examples of malignant tumors include, but are not limited to, bladder cancer, blood cancer, bone cancer, brain cancer/central nervous system cancer, head and neck cancer, breast cancer, cervical cancer, colon cancer, duodenal cancer, esophageal cancer, eye cancer, gallbladder cancer, heart cancer, kidney cancer, larynx cancer, liver cancer, lung cancer, oral cancer, ovarian cancer, thyroid cancer, pancreatic cancer, pharyngeal cancer, prostate cancer, rectal cancer, stomach cancer, testicular cancer, uterine cancer, skin cancer, and AIDS-related cancer, Hodgkin's disease, lymphoma (including Hodgkin lymphoma and non-Hodgkin lymphoma), multiple myeloma, melanoma, leukemia (including lymphocytic leukemia, hairy cell leukemia, acute myeloid leukemia), choriocarcinoma, rhabdomyosarcoma, neuroblastoma, etc. “Anti-tumor” refers to resisting, inhibiting or eliminating tumors, and may also be described as “treating tumors”, “anti-cancer” or “treating cancer” in the present application. “Anti-tumor drug”, as used herein, includes drugs that prevent or treat tumor growth and metastasis. “Treatment” of a subject means any type of intervention or treatment of a subject with the purpose of reversing, alleviating, ameliorating, inhibiting, slowing down or preventing the occurrence, progression, development, severity or recurrence of symptoms, complications, disorders or biochemical indicators associated with a disease. The anti-tumor drug promotes tumor regression or even elimination in the subject. “Promote tumor regression” means that a therapeutically effective amount of the drug, administrated alone or in combination with an anti-tumor agent, results in a decrease in tumor growth or size, tumor necrosis, a decrease in the severity of at least one disease symptom, an increase in the frequency and duration of disease-free periods, or prevention of impairment or disability resulting from the disease, or ameliorates a patient's disease symptoms in other ways. Further, the terms “effective” and “effectiveness” with respect to a treatment include both pharmacological effectiveness and physiological safety. Pharmacological effectiveness refers to the ability of a drug to promote tumor regression in a patient. Physiological safety refers to the level of toxicity or other adverse physiological effects (adverse effects) at the cellular, organ and/or organismal level resulting from the administration of a drug.

“Therapeutically effective dose” is any amount of a drug, as described below, that, when used alone or in combination with another therapeutic agent, promotes regression of disease manifested by a decrease in the severity of disease symptoms, an increase in the frequency and duration of disease-free periods, or prevention of impairment or disability resulting from the disease. The therapeutically effective amount or dose of a drug includes a “prophylactically effective amount” or a “prophylactically effective dose”, which is any amount of a drug that is described below, This amount of drug, when administered alone or in combination with another therapeutic agent to a subject at risk of developing the disease or suffering recurrence of the disease, may inhibit the occurrence or recurrence of the disease. The ability of a drug or therapeutic agent to promote regression of disease or to inhibit progression or recurrence of disease can be assessed in a variety of ways known to the skilled artisan, for example, by testing the activity of the agent in clinical trials in human subjects, in animal model systems that are predictive of efficacy in humans, or in an in vitro assay system.

“Pharmaceutically acceptable salt” refers to toxicologically compatible organic or inorganic salts of active molecules. Example salts include, but are not limited to: sulfates, citrates, acetates, oxalates, chlorine bromide, iodides, nitrates, bisulfates, phosphates, acid phosphates, isonicotinates, lactates, salicylates, citrates, tartrates, oleates, tannates, pantothenates, tartrates, ascorbates, succinates, maleates, gentisates, fumarates, gluconates, glucuronates, formates, benzoates, glutamates, methanesulfonic acid “methanesulfonate”, ethanesulfonates, benzenesulfonates, alkali metal (such as sodium and potassium) salts, alkaline earth metal (such as magnesium) salts, and ammonium salts. A pharmaceutically acceptable salt may have one or more charged atoms and/or one or more counter ions. If the active molecule is an alkali, the pharmaceutically acceptable salt can be prepared by treating the free alkali with an acid by conventional chemical methods. Such acids include inorganic acids such as hydrochloric acid, hydrobromic acid, sulfuric acid, nitric acid, methylsulfonic acid, and phosphoric acid, or organic acids such as acetic acid, maleic acid, succinic acid, mandelic acid, fumaric acid, malonic acid, pyruvic acid, oxalic acid, glycolic acid, salicylic acid, uronic acid (such as glucuronic acid or galacturonic acid), alpha hydroxy acid, citric acid, tartaric acid, amino acid (such as aspartic acid, glutamic acid), aromatic acid (such as benzoic acid or cinnamic acid), sulfonic acid (such as p-toluenesulfonic acid or ethanesulfonic acid), etc. If the active molecule is an acid, the desired pharmaceutically acceptable salt can be prepared by suitable methods using an inorganic or organic alkali such as ammonia, amine, alkali metal hydroxide or alkaline earth metal hydroxide. Examples of suitable salts include, but are not limited to, amino acid salts (such as glycine and arginine), ammonium salts, primary amine salts, secondary amine salts, tertiary amine salts, cyclic amines (such as piperidine, morpholine and piperazine), sodium salts, calcium salts, potassium salts, magnesium salts, manganese salts, iron salts, copper salts, zinc salts, aluminum salts and lithium salts.

The metabolites of the compounds of the present application and pharmaceutically acceptable salts of the compounds, as well as prodrugs that can be converted into the structures of the compounds of the present application and their pharmaceutically acceptable salts in vivo, are also included in the claims of the present application.

The active substance involved in the present application may be administered to mammals, preferably humans, alone or in combination with a pharmaceutically acceptable carrier in a pharmaceutical composition, according to standard pharmaceutical techniques. It can be administered orally or subcutaneously, intramuscularly, intraperitoneally, intravenously, rectum and topically, to the eyes, lungs, and nasal cavity and parenterally.

“Pharmaceutically acceptable carrier” refers to one or more excipients, stabilizers, fillers, binders, humectants, disintegrants, solution retardants, absorption enhancers, wetting agents, absorbents, lubricants, colorants, diluents, emulsifiers, preservatives, solubilizers, suspending agents, etc. These carriers may be administered to subjects at doses and concentrations commensurate with a reasonable benefit/risk ratio and without undue adverse side effects (e.g., toxicity, irritation, and allergic reactions). Examples of the pharmaceutically acceptable carrier include water, citrate or phosphate buffer, starch, lactose, sucrose, glucose, mannitol, carboxymethylcellulose, alginate, gelatin, polyvinylpyrrolidone, glycerin, agar, calcium carbonate, alginic acid, sodium carbonate, paraffin, quaternary ammonium compounds, cetyl alcohol, glyceryl monostearate, kaolin and bentonite, talc, calcium stearate, magnesium stearate, polyethylene glycol, sodium lauryl sulfate, ethanol, isopropyl alcohol, ethyl carbonate, ethyl acetate, benzyl alcohol, benzyl benzoate, propylene glycol, 1,3-butanediol, oil, tetrahydrofuran alcohol, fatty acid ester, sulfide isostearyl alcohol, polyoxyethylene sorbitol and sorbitol ester, microcrystalline cellulose, aluminum metahydroxide, tragacanth, and mixtures thereof, as well as other ingredients well known to those skilled in the art.

The active substance of the present application may be used in combination with one or more other drugs known to treat or ameliorate similar symptoms of a disease. In a case of combined administration, the original administration mode and dosage of the drug may remain unchanged, while the drug prepared from the active substance of the present application is administered at the same time or subsequently. When the drug prepared from the active substance of the present application is administered concomitantly with one or more other drugs, it is preferred to use a pharmaceutical composition containing both one or more known drugs and the drug prepared from the active substance of the present application. Drug combination also include administration of the drug prepared from the active substance of the present application and one or more other known drugs in overlapping time periods. When the drug prepared from the active substance of the present application is administrated in combination with one or more other drugs, the dose of the drug prepared from the active substance of the present application or the doses of known drugs may be lower than their doses when they are administrated alone.

Drug or active ingredients that can be used in combination to treat tumors include, but are not limited to: estrogen receptor modulators, androgen receptor modulators, retinoid receptor modulators, cytotoxic/cytokine inhibitors, antiproliferative agents, protein transferase inhibitors, HMG-COA reductase inhibitors, HIV protein kinase inhibitors, reverse transcriptase inhibitors, angiogenesis inhibitors, cell proliferation and survival signal inhibitors, drugs that interfere with cell cycle checkpoints and apoptosis inducers, cytotoxic drugs, tyrosine protein inhibitors, EGFR inhibitors, VEGFR inhibitors, serine/threonine protein inhibitors, Bcr-Abl inhibitor, c-Kit inhibitors, Met inhibitors, Raf inhibitors, MEK inhibitors, MMP inhibitors, topoisomerase inhibitors, histidine deacetylase inhibitors, proteasome inhibitors, CDK inhibitors, Bcl-2 family protein inhibitors, MDM2 family protein inhibitors, IAP family protein inhibitors, STAT family protein inhibitors, PI3K inhibitors, AKT inhibitors, integrin blockers, interferon-α, interleukin-12, COX-2 inhibitors, p53, p53 activators, VEGF antibodies, EGF antibodies, etc.

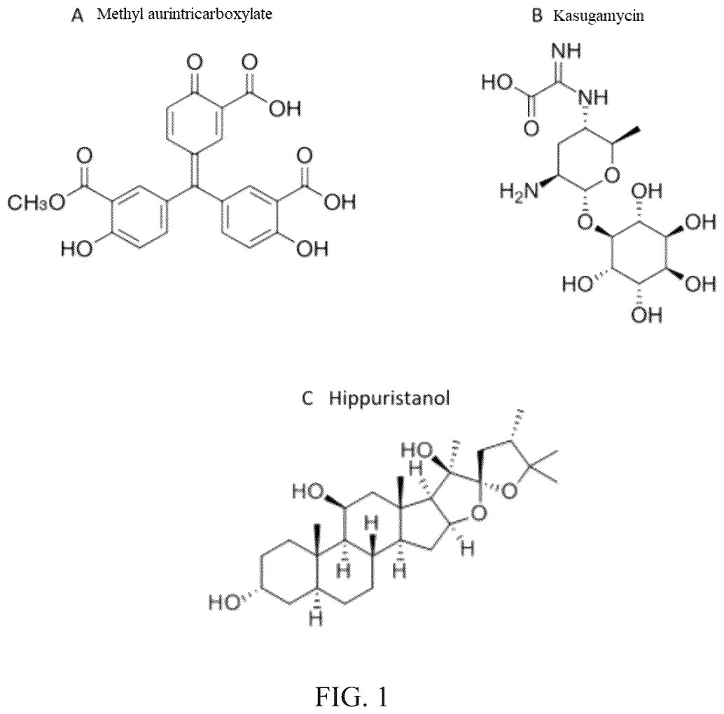

The chemical structural formulas of methyl aurintricarboxylate, Kasugamycin and Hippuristanol are shown in FIG. 1.

Methyl aurintricarboxylate is synthesized by standard Steglich esterification reaction between the raw material aurine tricarboxylic acid and methanol in the presence of catalysts DCC and DMAP, or by modified Steglich esterification reaction in the presence of catalysts EDC and DMAP. The reaction may be carried out at room temperature or by heating to 45° C. to improve the reaction rate. If a high-purity product is required, HPLC may be used for purification and separation. DCC refers to N,N′-Dicyclohexylcarbodiimide. DMAP refers to 4-Dimethylaminopyridine. EDC refers to 1-ethyl-3-(3-dimethylaminopropyl)-carbodiimide hydrochloride.

Kasugamycin and Hippuristanol are both purchased.

Kasugamycin: CAS RN: 6980-18-3; Supplier: Beijing Baiaolaibo Technology Co., Ltd.; Cat. No.: Y15224.

Hippuristanol: CAS RN: 80442-78-0; Supplier: Hangzhou RaystarBio Co., Ltd.; Cat. No.: GY05894.

The technical solution of the present application will be clearly and completely described below with reference to specific embodiments of the present application. Obviously, the described embodiments are only some of the embodiments of the present application, rather than all of the embodiments. All other embodiments obtained by those of ordinary skill in the art based on the embodiments of the present application without creative efforts shall fall within the scope of the present application.

### Example 1 Preparation of Methyl Aurintricarboxylate

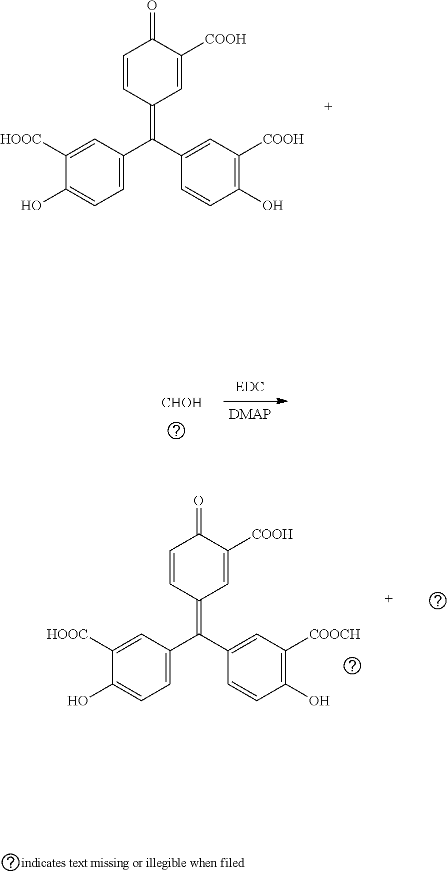

To 15 mL of acetonitrile, added were 1 mmol of aurintricarboxylic acid (CAS RN: 4431-00-9; Supplier: Sinopharm Chemical Reagent Co., Ltd.; Cat. No.: LA1590501), 2.55 mmol of DMAP, 1.28 mmol of EDC, and 0.85 mmol of methyl alcohol. The resulting solution was well mixed. The reaction was left overnight at room temperature. After the reaction was completed, the acetonitrile was evaporated under reduced pressure, and a solid crude product was obtained. The crude product was extracted with 20 mL of diethyl ether (or ethyl acetate), then washed twice with 20 mL of 1M HCl, then washed twice with 20 mL of saturated Na₂CO₃ solution, and then dried with anhydrous Na₂SO₄. Diethyl ether (or ethyl acetate) was removed under reduced pressure in a rotary evaporator. Methyl aurintricarboxylate (0.77 mmol, yield 77%) was obtained. LC-MS (m/z): 437.38[M]⁺.

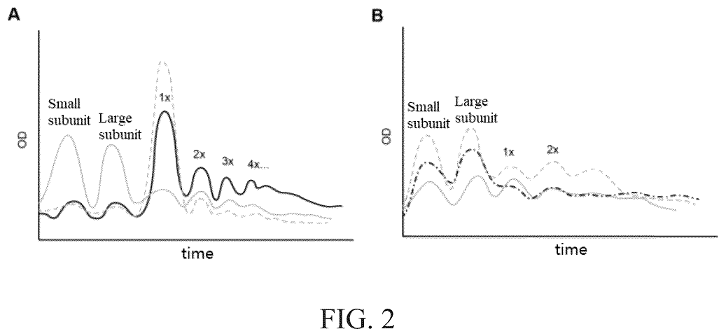

Example 2 Proof that methyl aurintricarboxylate, Kasugamycin and Hippuristanol are all translation inhibitors without occupying ribosome resources Using methods well known to those skilled in the art, sucrose density ultracentrifugation (polysome profiling) was carried out to measure polysomes. Ultracentrifugation was performed on normal cell lysates and drug-treated cell lysates at 15-50% sucrose density gradient, the solution was pumped out slowly, and OD260 nm (260 nm Optical Density) was measured. The results are shown in FIG. 2. In FIG. 2A, the black line, gray solid line and gray dashed line are the experimental curves of normal cells, cells treated with methyl aurintricarboxylate (concentration 50 μg/mL) and cells treated with Harringtonin (concentration 10 μg/mL) respectively. In FIG. 2B, the gray solid line, gray dashed line, and black dotted line are the experimental curves of cells treated with Kasugamycin (concentration 10 μg/mL), Pateamine A (concentration 2 μM), and Hippuristanol (concentration 1 μM), respectively. The peak heights of the curves are related to concentration.

Normal translation is going on in normal cells, so peaks of 40S, 60S, 80S (single ribosomes, marked as 1× in the figure), 2× ribosomes, 3× ribosomes and the like can be seen. Normally, more than 80% of ribosomes in the cells are in translation, so the peaks of 40S and 60S are relatively low. Under the action of aurintricarboxylic acid, Kasugamycin, Pateamine A or Hippuristanol, the peaks of 40S and 60S dominate and the peaks of complete ribosomes such as 1× and 2× are very small, indicating that the translation initiation is suppressed as a whole, and the large and small subunits of the ribosome cannot be successfully assembled into a complete ribosome. This result can prove that aurintricarboxylic acid, Kasugamycin, Pateamine A or Hippuristanol does not occupy ribosome resources and they are translation inhibitors without occupying ribosome resources. After the drug is removed by virtue of dialysis, sucrose density gradient centrifugation, etc., appropriate buffer components are added, and translation then can proceed normally again.

The control drug Harringtonin and other drugs will get the ribosome stuck at the translation initiation site and cannot enter the elongation phase of translation, so the peak of 1× ribosomes is very high and the peak of polysomes is very small (ribosomes that have entered the elongation phase of translation are not controlled by Harringtonin, and given enough time, they will complete translation and fall off), and the large and small subunit peaks of 40S and 60S are also very small. This shows that although Harringonin also inhibits translation initiation, it will occupy a large amount of ribosome resources. These assembled 80S ribosomes are stuck at the translation initiation site and cannot perform translation functions, nor can they simply dissociate into large and small subunits, thus occupying ribosome resources.

### Example 3 Anti-Tumor Activity Test of Methyl Aurintricarboxylate on Lung Cancer Cells

Cell migration, cell invasion, cell apoptosis, cell colony formation and cytotoxicity experiments were carried out using methods well known to those skilled in the art.

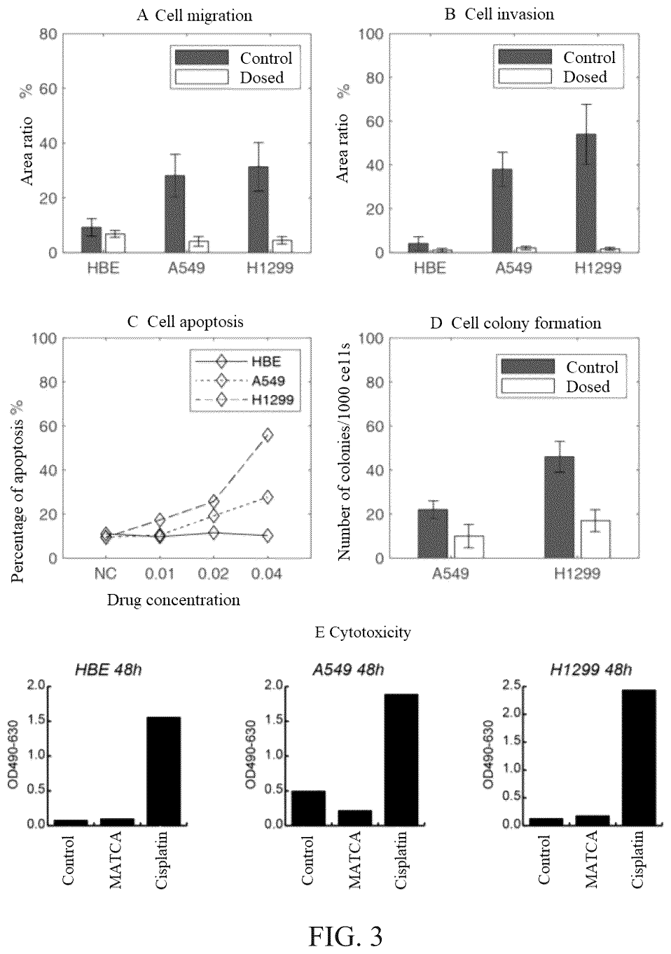

The selected cell lines: normal human bronchial epithelial cell (HBE), and two lung adenocarcinoma cells with different malignant degrees (A549 and H1299). The test procedure was as follows: (a) the above cells were incubated in a cell culture medium at 37° C., 0.5% CO₂. (b) The active substance to be tested (methyl aurintricarboxylate or cisplatin) was added, the cell solution was shaken well, and the cells were further incubated in the original environment. (c) The migration, invasion, apoptosis, colony formation and cytotoxicity of the obtained cells were tested. The test results are shown in FIG. 3.

(1) Cell migration: The Transwell migration assay was carried out to test the migration ability of HBE, A549 and H1299 cells passing pores before and after treatment. The final concentration of methyl aurintricarboxylate was 20 μg/mL. As shown in FIG. 3A, compared with the blank control, methyl aurintricarboxylate has little effect on normal cells (HBE), but significantly inhibits the metastasis ability of cancer cells (A549 and H1299).

(2) Cell invasion: Transwell invasion assay was carried out on HBE, A549 and H1299 cells treated with or without methyl aurintricarboxylate (final concentration 20 μg/mL). As shown in FIG. 3B, compared with the blank control, methyl aurintricarboxylate significantly inhibits the invasion ability of cancer cells (A549 and H1299).

(3) Cell apoptosis: Cells were fluorescently labeled using Annexin V, and the occurrence of cell apoptosis was tested under a fluorescence microscopy or on a flow cytometer. As shown in FIG. 3C (the relationship curve of the percentage of cell apoptosis over the concentration of methyl aurintricarboxylate), the addition of methyl aurintricarboxylate will not induce apoptosis in normal cells (HBE), but will induce apoptosis in cancer cells (A549 and H1299). As the concentration of methyl aurintricarboxylate increases (final concentration: 10 μg/mL, 20 μg/mL, 40 μg/mL), the percentage of apoptosis of cancer cells increases. The percentage of apoptosis of cancer cells is positively correlated with the concentration of methyl aurintricarboxylate. Under the same experimental conditions, the onset concentration of aurintricarboxylic acid (0.25-0.5 mg/mL) is 12.5 to 25 times that of methyl aurintricarboxylate.

(4) Cell colony formation: Agar colony formation assay was carried out on A549 and H1299 cells that were untreated and treated with methyl aurintricarboxylate (final concentration 20 μg/mL). As can be seen from FIG. 3D, the number of colonies/1000 cells of cancer cells (A549 and H1299) was significantly reduced after treatment with methyl aurintricarboxylate, indicating that the stemness of cancer cells and their ability to colonize after metastasis are inhibited.

(5) Cytotoxicity: 48 h after the addition of methyl aurintricarboxylate (final concentration 20 μg/mL) and cisplatin (50 μM) to the HBE, A549 and H1299 cells, the cytotoxicity was tested by LDH (lactate dehydrogenase) assay.

The concentration of cisplatin was determined according to the reported procedure (Fang, C. et al. MiR-488 inhibits proliferation and cisplatin sensibility in non-small-cell lung cancer (NSCLC) cells by activating the eIF3a-mediated NER signaling pathway. Sci. Rep. 7, 2017, 40384; Yang X. et al. ACTL6A promotes repair of cisplatin-induced DNA damage, a new mechanism of platinum resistance in cancer, PNAS, January 2021, 118 (3), e2015808118), and the concentration that inhibited lung cancer A549 cells by 80-90% was adopted.

This assay measured the difference in optical density at 490 nm and 630 nm. The greater the difference, the greater the cytotoxicity. It can be seen from FIG. 3E that although cisplatin can effectively kill the two lung cancer cells A549 and H1299 at a concentration of 50 μM, it can also effectively kill normal cells (HBE), thus showing high cytotoxicity. In contrast, methyl aurintricarboxylate has little cytotoxicity, so it can be seen that methyl aurintricarboxylate is highly safe.

The above test results (1) to (5) show that methyl aurintricarboxylate not only has good inhibition and killing effects on cancer cells, but also has almost no effect on normal cells and is highly safe.

### Example 4 Anti-Tumor Activity Test of Methyl Aurintricarboxylate on Ovarian Cancer Cells

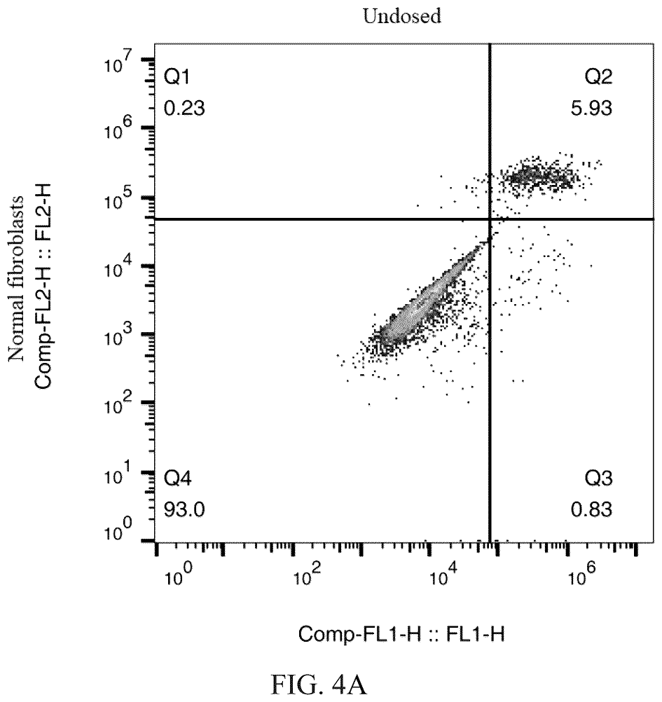

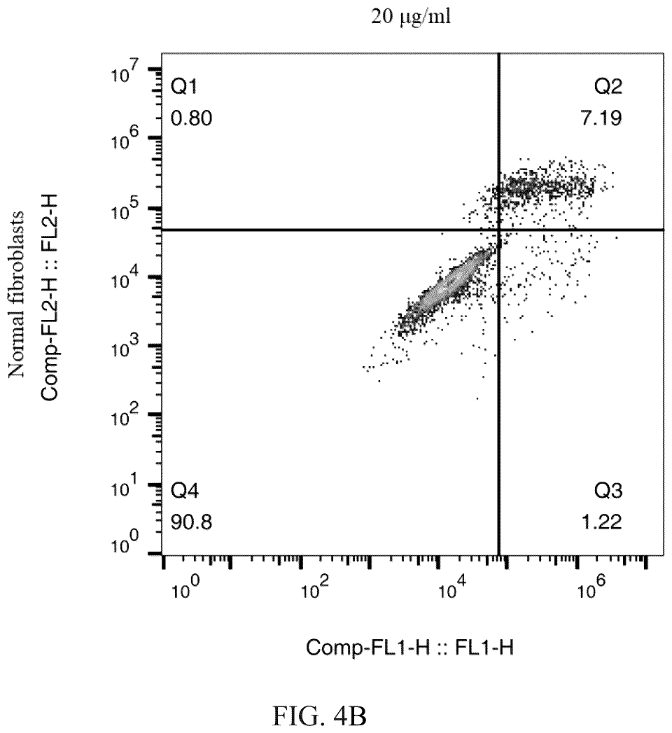

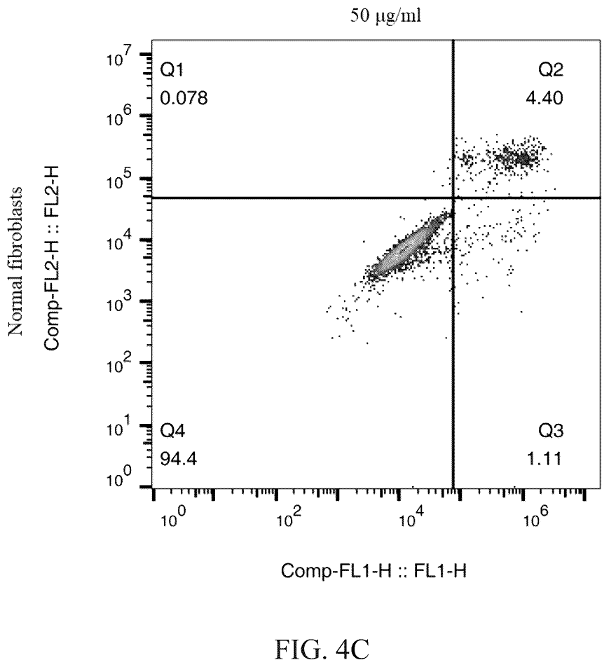

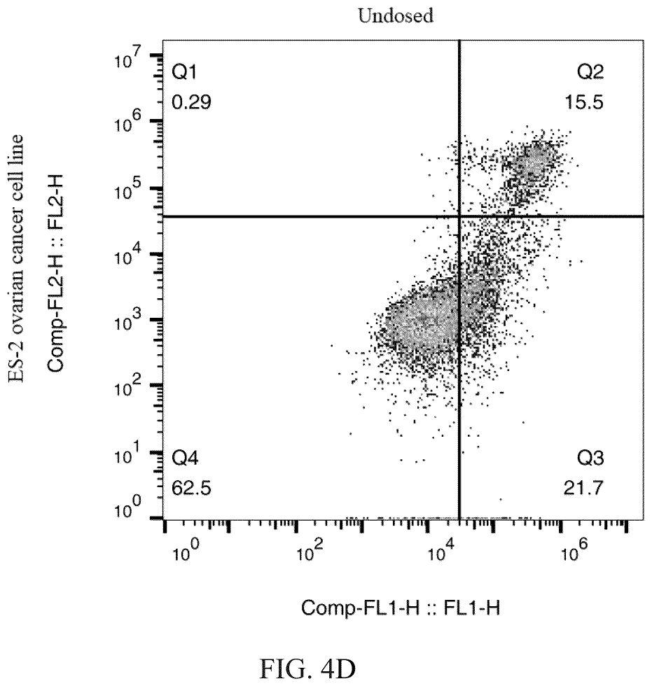

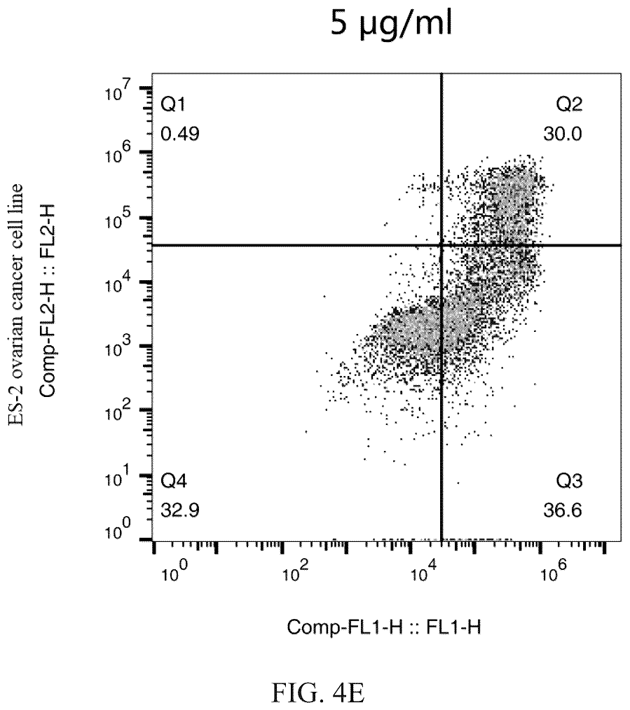

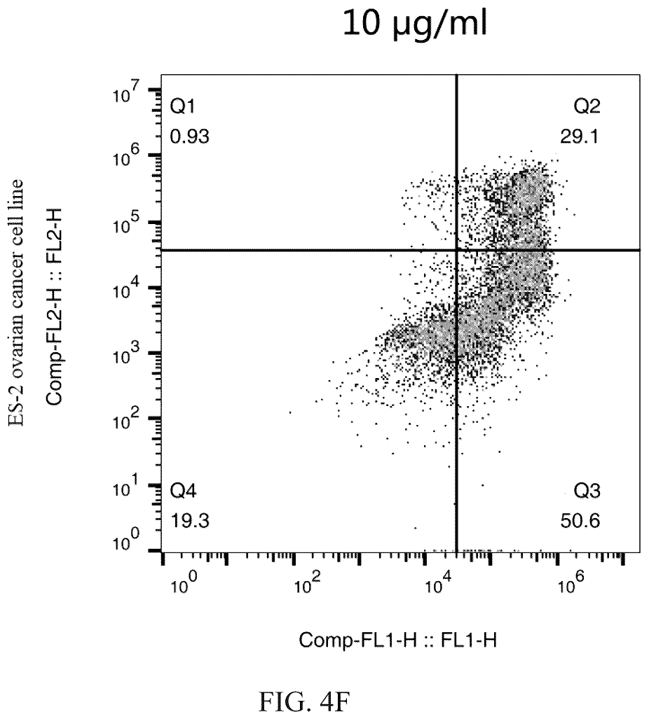

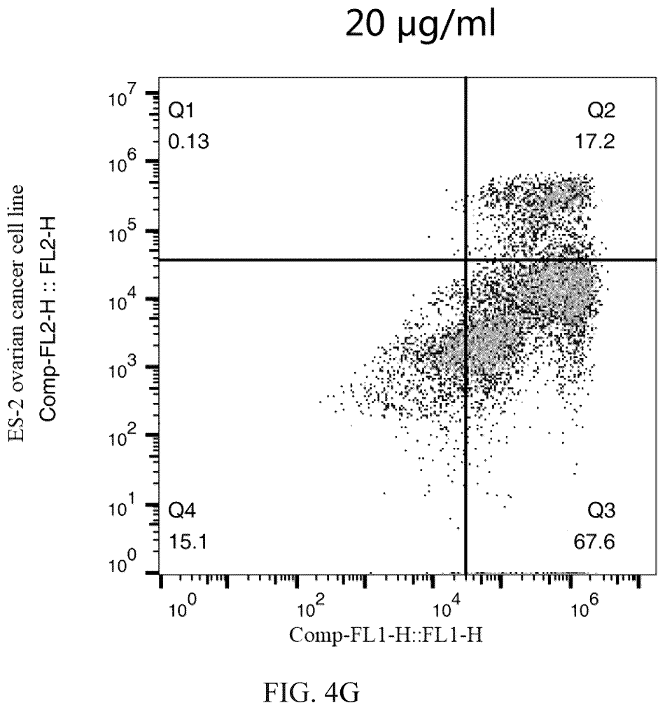

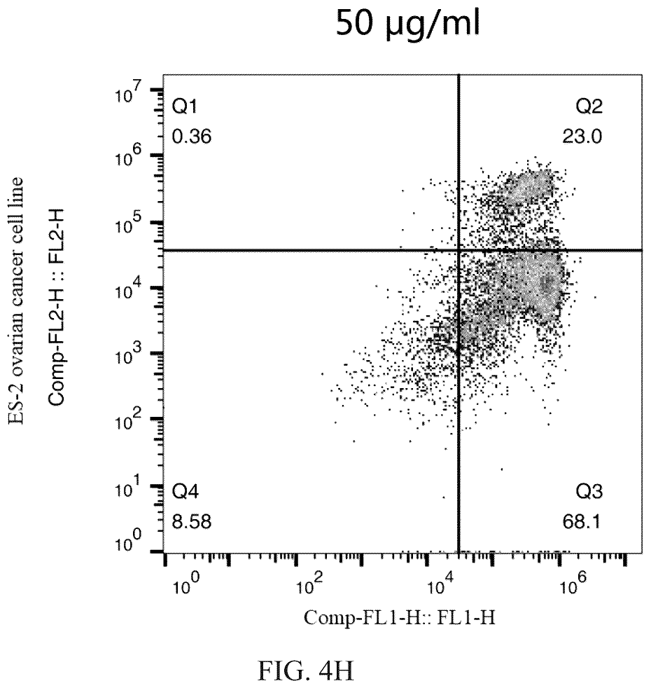

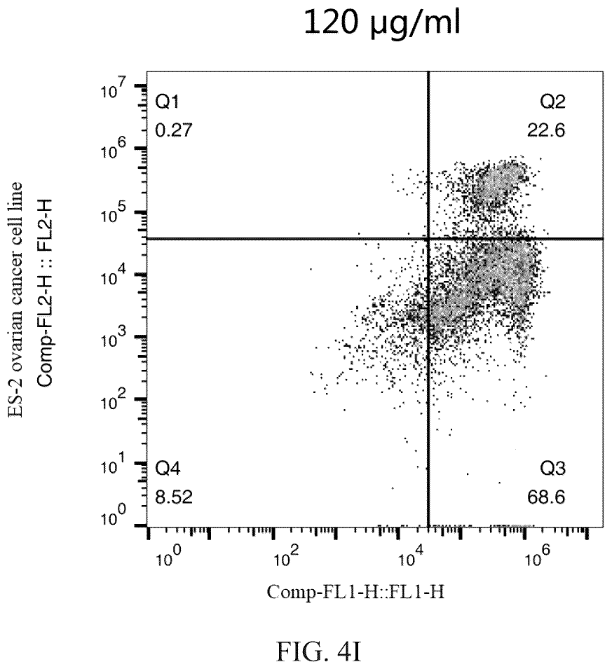

Apoptosis assay was carried out using human normal fibroblasts (primary culture) as normal controls and ES-2 human ovarian cancer cell line as cancer cells. The test procedure was as follows: (a) the above cells were incubated in a cell culture medium at 37° C., 0.5% CO₂. (b) A methyl aurintricarboxylate solution was added, the cell solution was shaken well, and the cells were further incubated in the original environment for 48 h. (c) Apoptosis was tested by Annexin V flow cytometry assay. The results are shown in FIGS. 4A-4I. In the figure, cells in the area Q1 are non-apoptotic cells, cells in the area Q3 are early apoptotic (the key area for observing apoptosis), and cells in the area Q2 are late apoptotic, including some necrotic cells.

As can be seen from FIGS. 4A-4I, when the dose of methyl aurintricarboxylate is within a range of 20 μg/mL to 50 μg/mL, it has basically no effect on normal cell apoptosis. When the dose is 5 μg/mL, it has a significant effect on promoting apoptosis in the ES-2 cell line. When the dose is 20 μg/mL, early apoptosis has reached 67.6%. At higher concentrations, almost all cells are completely apoptotic.

In addition, when the dose of methyl aurintricarboxylate is 20 μg/mL or above, late apoptotic+necrotic cells do not increase significantly, and there is no obvious dose-effect relationship (see Table 1 for specific data). This shows that methyl aurintricarboxylate does not significantly cause necrosis, which plays an important role in avoiding exacerbation of the tumor inflammatory microenvironment.

TABLE 1
The dosage of methyl aurintricarboxylate and the
percentage of late apoptotic and necrotic cells

| | Dose of methyl aurintricarboxylate | | | | |
|---|---|---|---|---|---|
| | 0 | 10 μg/mL | 20 μg/mL | 50 μg/mL | 120 μg/mL |
| Percentage | 15.5% | 29.1% | 17.2% | 23.0% | 22.6% |
| of late | | | | | |
| apoptotic | | | | | |
| and necrotic | | | | | |
| cells | | | | | |

### Example 5 Anti-Tumor Activity Test of Methyl Aurintricarboxylate on Liver Cancer Cells

The Chinese liver cancer cell lines HCCLM3 and MHCC97H were taken, and cell proliferation was tested with the addition of methyl aurintricarboxylate. The test procedure was as follows (a) HCCLM3 and MHCC97H9 cells were incubated in a cell culture medium at 37° ° C., 0.5% CO₂. (b) A methyl aurintricarboxylate solution was added to the culture medium and the final concentrations of methyl aurintricarboxylate reached 10 μg/mL, 20 μg/mL, 50 μg/mL, 80 μg/mL and 120 μg/mL, respectively. The cells were further incubated for 48-84 before sampling. (c) Cell proliferation was tested by CCK-8 (Cell Counting Kit-8).

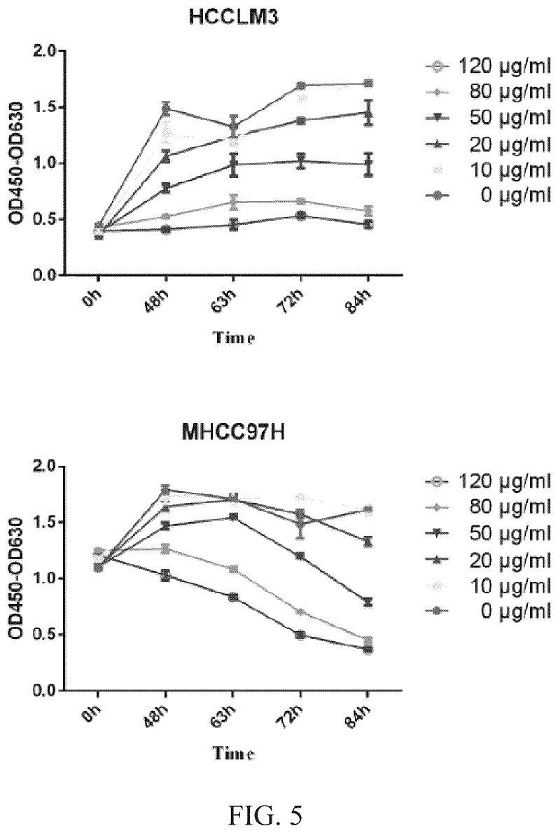

The proliferation results of HCCLM3 and MHCC97H cells before and after treatment are shown in FIG. 5. It can be seen from the figure that as the drug concentration increases, the proliferation rate of cancer cells decreases significantly, indicating that the inhibitory effect of methyl aurintricarboxylate on the proliferation of the two liver cancer cell lines shows a dose-effect relationship, methyl aurintricarboxylate has obvious inhibitory effect at the concentration of 50 μg/mL or above.

### Example 6 Anti-Tumor Activity Test of Kasugamycin on Breast Cancer Cells

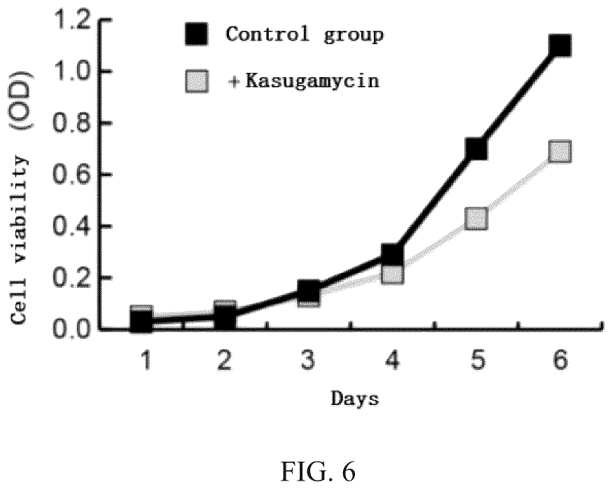

The human breast cancer cell line MDA-MB-468 was cultured, 1 mg/L Kasugamycin was then added, and cell proliferation was measured by the sulforhodamine B assay well known in the art (see Qin et al., BAPI promotes breast cancer cell proliferation and metastasis by deubiquitinating KLF5, Nature Communications 6:8471). As shown in FIG. 6, following the addition of Kasugamycin, cell proliferation slows down significantly from day 4, indicating that Kasugamycin has the ability to inhibit the proliferation of breast cancer cells.

### Example 7 Anti-Tumor Activity of Kasugamycin in a Nude-Mouse Lung Cancer Xenograft Model

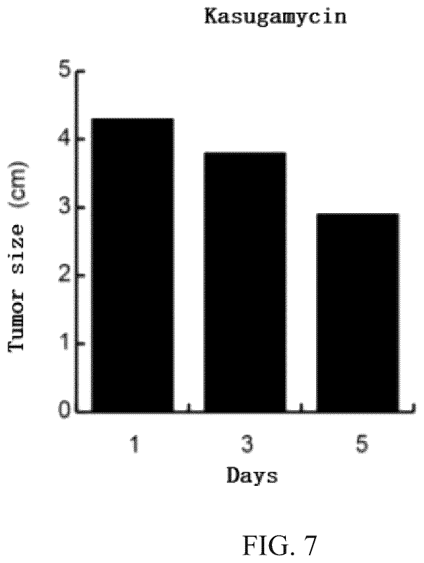

Using techniques well known in the art, the tumor inhibition assay of Kasugamycin was carried out on a nude-mouse xenograft model. Nude mice were subcutaneously injected with H1299 lung cancer cells. After the tumors formed, kasugamycin was administered (by gavage) once at a dose of 10 μg/kg. On days 1, 3, and 5, the tumor diameters of the nude mice were measured in vivo. The results of the change in tumor diameter are shown in FIG. 7. It can be seen that the tumor shrink significantly, indicating that Kasugamycin has an obvious anti-tumor effect.

### Example 8 Anti-Tumor Activity Test of Hippuristanol on Breast Cancer Cells

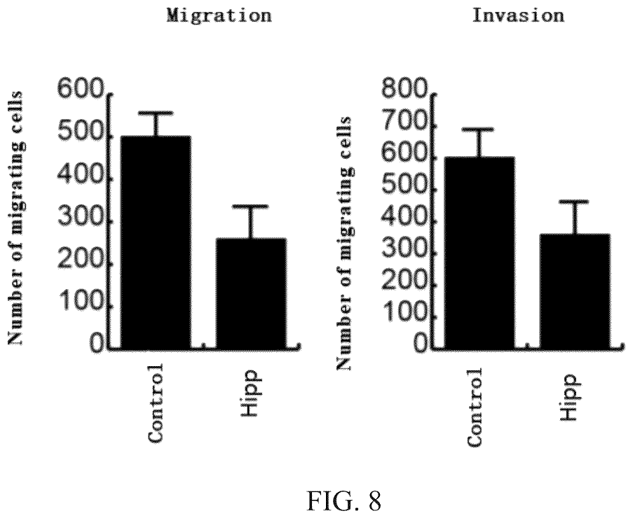

The human breast cancer cell line MDA-MB-468 was incubated and 50 nM Hippuristanol was then added. The well-known Transwell migration and invasion assays were carried out. 100,000 cells were added to the Transwell chamber, migration/invasion was carried out for 24 h, and crystal violet staining was performed. As can be seen from FIG. 8, 50 nM Hippuristanol can effectively reduce the migration and invasion of breast cancer cells.

### Example 9 Anti-Tumor Activity of Hippuristanol in a Nude-Mouse Lung Cancer Xenograft Model

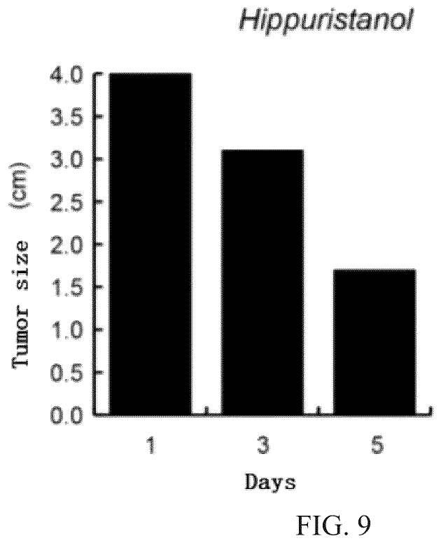

Using techniques well known in the art, the tumor inhibition assay of Hippuristanol was carried out on a nude-mouse xenograft model. Nude mice were subcutaneously injected with A549 lung cancer cells. After the tumors formed, Hippuristanol was administered by gavage once at a daily dose of 10 mg/kg. On days 1, 3, and 5, the tumor diameters of the nude mice were measured in vivo. The results of the change in tumor diameter are shown in FIG. 9. It can be seen that the tumor shrink significantly, indicating that Hippuristanol has an obvious anti-tumor effect.

The translation inhibitor without occupying ribosome resources according to the present invention not only has excellent tumor inhibition or killing effects, but also has superior safety and is an excellent anti-tumor drug molecule.

The above descriptions are only preferred embodiments of the invention, and are not intended to limit the invention in other forms. Any skilled person familiar with the art may make use of the technical content disclosed above to change or modify them into equivalent embodiments with equivalent changes. However, any simple modifications, equivalent changes and variants made to the above embodiments based on the technical essence of the invention without departing from the content of the technical solution of the invention still fall within the scope of the technical solution of the invention.

What is claimed is:

1. A translation inhibitor, wherein the translation inhibitor is a translation inhibitor without occupying ribosome resources.

2. The translation inhibitor according to claim 1, wherein the translation inhibitor is selected from a group consisting of methyl aurintricarboxylate, Kasugamycin and Hippuristanol, as well as a prodrug of one of methyl aurintricarboxylate, Kasugamycin and Hippuristanol or one or more of pharmaceutically acceptable salts of one of methyl aurintricarboxylate, Kasugamycin and Hippuristanol.

3. The translation inhibitor according to claim 1, wherein the translation inhibitor is methyl aurintricarboxylate, Kasugamycin or Hippuristanol, or a prodrug of one of methyl aurintricarboxylate, Kasugamycin and Hippuristanol, or a pharmaceutically acceptable salt of one of methyl aurintricarboxylate, Kasugamycin and Hippuristanol.

4. The translation inhibitor according to claim 3, wherein the pharmaceutically acceptable salt is an ammonium salt, a sodium salt, a potassium salt or a hydrochloride salt.

5. Use of the translation inhibitor according to claim 1 in preparation of an anti-tumor drug.

6. The use according to claim 5, wherein a tumor to which the anti-tumor drug is applied is a malignant tumor.

7. The use according to claim 6, wherein the malignant tumor is one or more selected from a group consisting of lung cancer, liver cancer and ovarian cancer.

8. A pharmaceutical composition, comprising the translation inhibitor according to claim 1 and a pharmaceutically acceptable carrier.

9. The pharmaceutical composition according to claim 8, further comprising one or more anti-tumor active ingredients other than the translation inhibitor according to claim 1.
